(19) United States
(12) Patent Application Publication
(10) Pub. No.: US 2023/0346304 A1
(43) Pub. Date: Nov. 2, 2023

(54) Method for OSA Severity Detection Using Recording-based Electrocardiography Signal

(71) Applicant: National Yang Ming Chiao Tung University, Hsinchu (TW)

(72) Inventors: Sin Horng Chen, Hsinchu (TW); Cheng Yu Yeh, Hsinchu (TW); Chun Cheng Lin, Hsinchu (TW); Shaw Hwa Hwang, Hsinchu (TW); Yuan Fu Liao, Hsinchu (TW); Yih Ru Wang, Hsinchu (TW); Kuan Chun Hsu, Hsinchu (TW); You Shuo Chen, Hsinchu (TW); Yao Hsing Chung, Hsinchu (TW); Yen Chun Huang, Hsinchu (TW); Chi Jung Huang, Hsinchu (TW); Li Te Shen, Hsinchu (TW); Bing Chih Yao, Hsinchu (TW); Ning Yun Ku, Hsinchu (TW)

(21) Appl. No.: 17/732,844

(22) Filed: Apr. 29, 2022

(51) Int. Cl. A61B 5/00; A61B 5/318

(52) U.S. Cl. CPC A61B 5/4842; A61B 5/4818; A61B 5/6802; A61B 5/318; A61B 5/7267

(57) ABSTRACT

The present invention provides a method for OSA (Obstructive Sleep Apnea) severity detection using recording-based electrocardiography (ECG) Signal. The major feature of the present invention emphasizes on using a recording-based ECG Signal as an input, which is different from the deep learning-based prior art of using segment-based signals as an input to a model, and the segment-based signals has only two classification results, i.e. normal or apnea. The present invention provides a method for a model to detect and output directly a value of apnea-hypopnea index (AHI) for the OSA Severity.

### FIELD OF THE INVENTION

The present invention relates to a method for OSA (Obstructive Sleep Apnea) severity detection, and more particularly of using a recording-based electrocardiography (ECG) signal as an input to detect and output directly a value of apnea-hypopnea index (AHI) for the OSA Severity.

### BACKGROUND OF THE INVENTION

Referring to FIG. 1, which shows a deep learning technology of the prior art, using segment-based signals as inputs to differentiate, and only provide two recognition models for OSA, i.e. normal or apnea. A recording-based electrocardiography (ECG) signal 1 is segmented to K-segmented ECG signals 2, and fed respectively to the recognition model of two-category for OSA severity 3, and obtains results corresponding to the segmented ECG signal 4 (normal or apnea), then conduct OSA severity evaluation 5 to show outcome 6.

Using an example to describe the OSA severity evaluation in FIG. 1:

Suppose that a person's sleeping time is 8 hours, OSA severity evaluation can conduct throughout the 8 hours. However, 8 hours are not a fixed time, no limitation to the sleeping time, 8 hours are just an example.

Suppose: A recording-based ECG signal 1 has a time length of 8 hours;

A segmented ECG signal 2 has a time length T of 60 seconds;

Total cut K=480 segmented ECG signals 2 (K*T=28800 seconds=8 hours);

Suppose OSA severity evaluation 5 shows that L=200 segmented ECG signal 2 are apnea;

Therefore apnea-hypopnea index (AHI) is calculated, AHI=L/(K*T/3600), which means apnea times per hour:

Normal: AHI<5

Mild: 5≤AHI<15

Moderate: 15≤AHI<30

Severe: AHI≥30

In the above example, AHI=25, which means the result is a moderate case.

The above method has three disadvantages: the first is that the recognition process is very complicated; the second is that the accuracy of the recognition model is influenced by the different time length of the segment-based signals; the third is that the recognition model uses datasets of segment-based signals during training procedures, which has a very complicated labeling work, the cost of manpower and time is considerable.

### SUMMARY OF THE INVENTION

The object of the present invention is to provide a method for OSA (Obstructive Sleep Apnea) severity detection by using a recording-based electrocardiography (ECG) signal, the contents of the present invention are decribed as below.

Firstly a detection model of OSA severity is built up.

Acquire ECG signal from public datasets as the training material to input into the detection model of OSA severity for training, and achieve a model.

A recording-based whole ECG signal is inputted into the model for directly showing AHI value and a corresponding result of OSA severity (i.e. normal, mild, moderate or severe).

The recording-based whole ECG signal is inputted into the model, after a processing of a feature maps extraction layer based on convolutional neural network, a global average pooling layer, a dence layer and an output layer to obtain the AHI value and the corresponding result of four-category OSA severity.

### BRIEF DESCRIPTION OF THE DRAWINGS

FIG. 1 shows schematically the prior art of using segment-based signals as the input to the model, and provide a recognition model of two-category OSA severity.

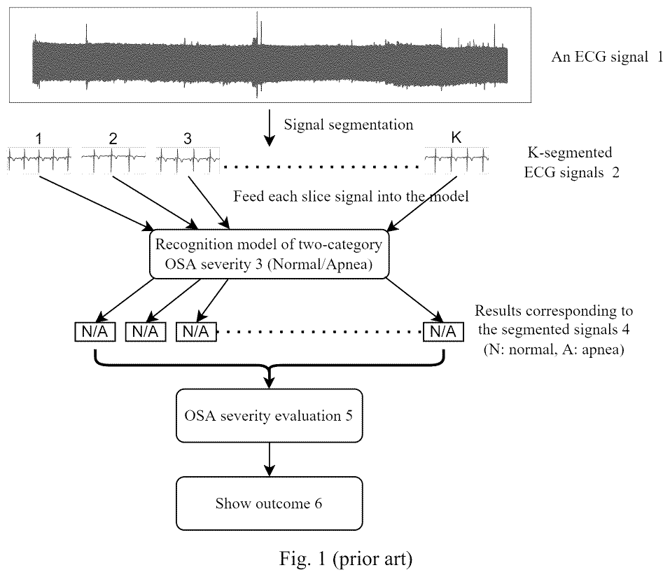

FIG. 2 shows schematically a recording-based electrocardiography (ECG) signal is used as input for detecting directly and showing AHI value and the corresponding category result of the OSA severity according to the present invention.

FIG. 3 shows schematically an embodiment of the detection model of the OSA severity in FIG. 2.

FIG. 4 shows schematically the training for generating the detection model of the OSA severity according to the present invention.

FIG. 5 shows schematically a participant wears on the hand a wearable device for measuring ECG signal and conducting OSA diagnosis according to the present invention.

### DETAILED DESCRIPTIONS OF THE PREFERRED EMBODIMENTS

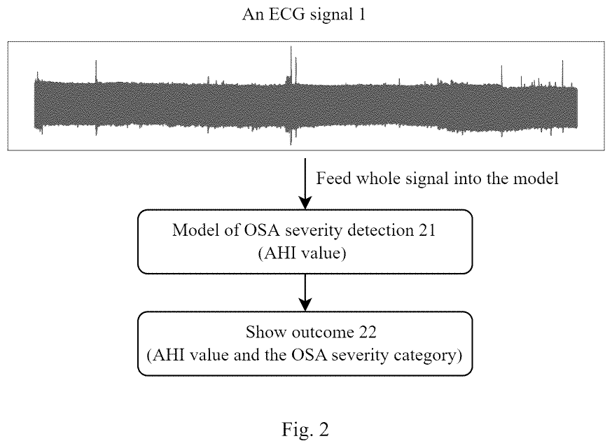

FIG. 2 describes that the present invention uses a recording-based ECG signal 1 as an input to detect directly AHI value of the OSA severity and the corresponding four-category result of the OSA severity and to show outcome 22 (AHI value and OSA severity category).

The present invention provides detection model of OSA severity 21 to output directly the AHI value and the corresponding result of the OSA severity, i.e. normal, mild, moderate or severe. A whole ECG signal 1 (for example 8 hours, but the time length is not limited) is inputed into the model for recognition and shows oucome 22 directly.

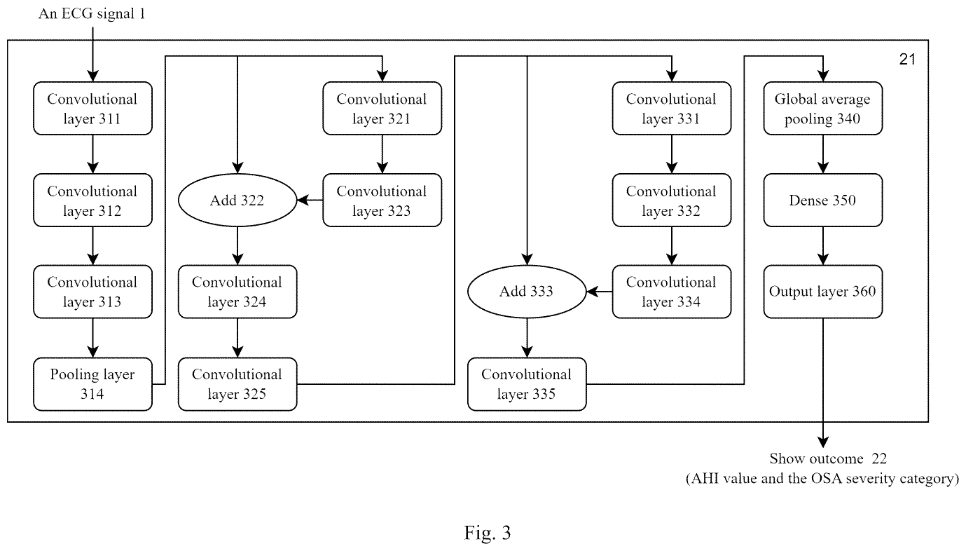

FIG. 3 describes in detail an embodiment of the detection model of OSA severity 21 in FIG. 2. The content in FIG. 3 can be divided into four parts, i.e. feature maps extraction layer based on convolutional neural network 311-335, global average pooling 340, dence 350 and output layer 360. The input signal of the present invention is a recording-based whole ECG signal 1, the time length of the input signal is not limited, any time length of a input signal is permitted, while the input signal of the prior art (recognition model of two-category for OSA severity) has a fixed time length.

The feature maps extraction layer based on convolutional neural network 311˜335: The feature maps extraction layer uses convolutional neural network (CNN) to conduct feature maps extraction for the input signal. The convolutional neural network is used very often in deep learnig. The biggest feature in CNN is to extract automatically feature information of the input signal through model training, which is called feature maps, and then the feature maps is used for conducting recognition. This method can promote the accuracy of recognition efficiently. The CNN is composed of convolution layers, activation functions and pooling layers. By using these layers to conduct multiple layers of parallel or series connection repeatedly, various CNNs can be built up. In the present embodiment, convolutional layer 311-313 and pooling layer 314 are the first level for feature maps extraction; convolutional layer 321, add 322, convolutional layer 323, convolutional layer 324, and convolutional layer 325 are the second level for feature maps extraction, while convolutional layer 331, convolutional layer 332, add 333, convolutional layer 334 and convolutional layer 335 are the third level for feature maps extraction.

The global average pooling 340: A global average pooling method is used for calculating an average value for each feature map as the ouput of the pooling layer. This method can convert an input signal of different length into an output signal of the same length. In other word, this method can make the model of the present invention accept input signal of any length.

The dence 350: to integrate the features of high abstraction obtained above, and then transfer to the output layer 360.

The output layer 360: use Rectified Linear Unit (ReLU) activation function to output an AHI value (≥0).

In the present embodiment, all convolutional layers use kernel of 1-dimension. Convolutional layer 311 uses 32 kernels of size 20, expressed as (32, 20). Convolutional layer 312 uses kernels of (64, 20); Convolutional layer 313 uses kernels of (128, 5); Convolutional layer 321 uses kernels of (128, 3); Convolutional layer 323 uses kernels of (128, 3); Convolutional layer 324 uses kernels of (64, 1); Convolutional layer 325 uses kernels of (128, 3). Convolutional layer 331 uses kernels of (128, 3); Convolutional layer 332 uses kernels of (128, 3); Convolutional layer 334 uses kernels of (128, 3); Convolutional layer 335 uses kernels of (64, 1).

In the present embodiment, if the input ECG signal 1 samples out 2,160,000 sampling points within 6 hours by 100 Hz sampling rate, then after a convolutional operation of the convolutional layer 311 to convert into 32 feature maps of size 108,000. This feature maps is a 2-dimension array, expressed as 108,000×32. Then the convolutional layer 312 conducts a convolutional operation to the feature maps outputted from the convolutional layer 311, so as to generate 5,400×64 feature maps. The convolutional layer 313 conducts a convolutional operation to the 5,400×64 feature maps so as to generate 1,080×128 feature maps. The pooling layer 314 uses a sliding window of size 2 to adopt max pooling for the feature maps outputted from the convolutional layer 313, so as to obtain 540×128 feature maps.

Thereafter the convolutional layer 321 conducts a convolutional operation to the feature maps outputted from the pooling layer 314 so as to generate 540×128 feature maps, and then after a convolutional operation by the convolutional layer 323 to generate 540×128 feature maps. Then the feature maps outputted from the pooling layer 314 and the feature maps outputted from the convolutional layer 323 will conduct addition operation at the add 322 to obtain a merged 540×128 feature maps. The convolutional layer 324 conducts a convolutional operation to the feature maps outputted from the add 322 to obtain 540×64 feature maps. The convolutional layer 325 will conduct convolutional operation to feature maps outputted from the convolutional layer 324 to obtain 540×128 feature maps.

Thereafter the convolutional layer 331 conducts a convolutional operation to the feature maps outputted from the convolutional layer 325 to generate 540×128 feature maps, continue in this way by convolutional operation of the convolutional layer 332 and convolutional layer 334 to maintain 540×128 feature maps. Then the feature maps outputted from the convolutional layer 325 and the feature maps outputted from the convolutional layer 334 will conduct addition operation at the add 333 to obtain a merged 540×128 feature maps. Finally the convolutional layer 335 conducts convolutional operation to the feature maps putputted from the add 333 to obtain 540×64 feature maps.

The global average pooling 340 in the present embodiment then uses global average pooling method to treat feature maps outputted from the convolutional layer 335 and obtains 64 feature maps average value. It's worth mentioning that the global average pooling method can make input of different length to be converted into output of the same length, therefore the input signal of the present invention model can be any length by this method. Thereafter the features outputted from the global average pooling 340 will be linked to the dence 350 having 16 neurons. The dence 350 is linked to the output layer 360 having only 1 neuron. The output layer 360 uses ReLU activation function to output an AHI value (2:0). Finally, the show outcome 22 will display the AHI value and a corresponding result of four-category OSA severity (i.e. normal, mild, moderate or severe).

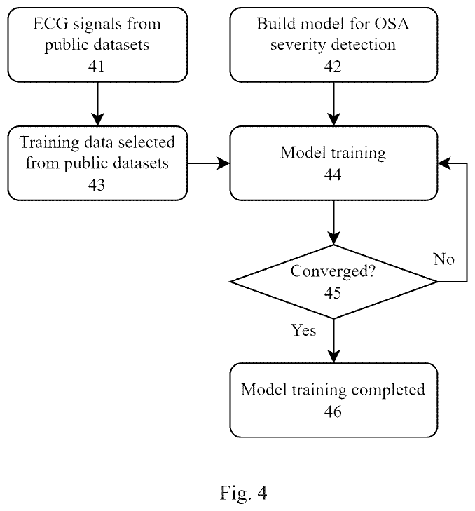

FIG. 4 describes how to train and generate a model according to the present invention. Firstly acquire ECG public datasets 41, build detection model of OSA severity 42, use ECG training data selected from public datasets 43 to input into OSA severity detection model for training 44. The model training will continue until model convergence is achieved 45. The completed model 46 can be used to conduct OSA diagnosis.

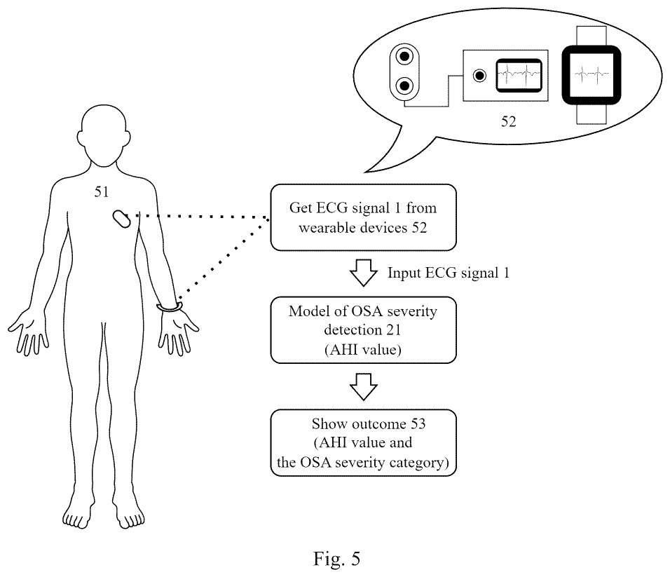

Nowadays wearable devices which can measure ECG signal are very popular, it is very convenient to conduct OSA diagnosis through ECG signal analysis, a user can do self-test at home. Referring to FIG. 5, a participant 51 wears on the hand a wearable device 52 for measuring ECG signal, and obtains ECG signal to input into the detection model of four-category OSA severity 21 for conducting OSA diagnosis, and show outcome 53 of the AHI value and corresponding diagnosis result of the OSA severity (normal, mild, moderate or severe) directly.

The scope of the present invention depends upon the following claims, and is not limited by the above embodiments.

What is claimed is:

1. A method for OSA (Obstructive Sleep Apnea) severity detection by using recording-based Electrocardiography (ECG) signal, comprising:
a. build up a detection model of OSA severity;
b. acquire ECG signals from public datasets to input into the detection model of OSA severity for training, and achieve a model;
c. a recording-based whole ECG signal is inputted into the model for directly showing an apnea-hypopnea index (AHI) value and a corresponding result of OSA severity (normal, mild, moderate or severe).

2. The method for OSA (Obstructive Sleep Apnea) severity detection by using recording-based Electrocardiography (ECG) signal according to claim 1, wherein the recording-based whole ECG signal is inputted into the model, after a processing of a feature maps extraction layer based on convolutional neural network, a global average pooling layer, a dence layer and an output layer to obtain the AHI value and the corresponding result of four-category OSA severity.

3. The method for OSA (Obstructive Sleep Apnea) severity detection by using recording-based Electrocardiography (ECG) signal according to claim 1, wherein a wearable device is used for obtaining the recording-based whole ECG signal.
